SEQUENCE LISTING (11 sequences)

SEQ ID NO: 1 - DNA, 885 bp, synthetic construct
  source 1..885: mol_type = other DNA
atggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactga

SEQ ID NO: 2 - DNA, 885 bp, Homo sapiens
  source 1..885: mol_type = other DNA
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaa

SEQ ID NO: 3 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 4 - DNA, 2050 bp, Homo sapiens
  source 1..2050: mol_type = genomic DNA
tcgaagctttataaaaacatacttttttttttacttttttttttttttctgagacacagcctcactctgtcgcccaggctggagtgcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgattttgtatttttaggagaaacagggtttcaccatgttgggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatattctgtcgcaaaaaaaaaaagaatactggaggctgggcgaggtggctcacacctgtaatcccagcattttgggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 5 - DNA, 928 bp, Homo sapiens
  source 1..928: mol_type = genomic DNA
ggatgccagaggcgggcggaatntcttgagctcaggagttcgagaccagcctacacaatatgctccaaacgccgcttntacaaaacatacagaaactacccgggtgtggtggcgnncccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgct

SEQ ID NO: 6 - DNA, 6286 bp, synthetic construct
  source 1..6286: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttcgaagctttataaaaacatacttttttttttacttttttttttttttctgagacacagcctcactctgtcgcccaggctggagtgcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgattttgtatttttaggagaaacagggtttcaccatgttgggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatattctgtcgcaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcttctacaaaacatacagaaactacccgggtgtggtggcgtgcccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctatggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactgagcggccgcaagcttcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 7 - DNA, 5198 bp, synthetic construct
  source 1..5198: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtggatgccagaggcgggcggaatatcttgagctcaggagttcgagaccagcctacacaatatgctccaaacgccgcttctacaaaacatacagaaactacccgggtgtggtggcgtgcccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctatggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactgagcggccgcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 8 - DNA, 4855 bp, synthetic construct
  source 1..4855: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcaccggtgccaccatggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactgagccaagcttcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 9 - DNA, 4926 bp, synthetic construct
  source 1..4926: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcaccggtgccaccatggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactgagcggccacaaacaccattgtcacactccaacaaacaccattgtcacactccaacaaacaccattgtcacactccaagcttcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 10 - DNA, 6352 bp, synthetic construct
  source 1..6352: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttcgaagctttataaaaacatacttttttttttacttttttttttttttctgagacacagcctcactctgtcgcccaggctggagtgcaggttttcatgtttatctgtgagatgtacctttggcacattactttcctgacatgagatttaaatttttttttttatcttgtgacaatttaacttttttgacacataaaaattgtacatatttatttgtttgagatggagtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgattttgtatttttaggagaaacagggtttcaccatgttgggccaggctggtcttgaagtcctgacctcaagtgatccacccaccttggcctcccaaagtgctgggattataggcatgagccaccgtaccagacccctaaaaattgtatatatttaaggtgtaccatttgatgtttagatatacattgtgaaatgattacattccacatattacctctacagagttaccatttttgtacacttggtcaacatcatcccattctccccttcctccacagatatttcttgtatactatatagaagccaagggtattttgggggaagagctcaaagttcctttcgtggagttaaaaatatatatatactatgtacatataagccatttagcaaccctagatgcttaataaagaatactggaggcccggtgtggtggctcacacctgtaatcccagcactttgggaggccgaggcggtcggattacgaggtcaggagttcaagaccagcctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatattctgtcgcaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcttctacaaaacatacagaaactacccgggtgtggtggcgtgcccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctatggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactgagcggccacaaacaccattgtcacactccaacaaacaccattgtcacactccaacaaacaccattgtcacactccaagcttcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgataactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattag

SEQ ID NO: 11 - DNA, 5264 bp, synthetic construct
  source 1..5264: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtggatgccagaggcgggcggaatatcttgagctcaggagttcgagaccagcctacacaatatgctccaaacgccgcttctacaaaacatacagaaactacccgggtgtggtggcgtgcccctgtggtcctagatacttgggaggttgaggcgggaggatcgcttgagctcgggaggtcgaggctgcaatgagccgagatggtgccactgcattctgacgacagagcgagattccgtttcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacacacacacacacacacacaccacactggagttcgagacgaggcctaagcaacatgccgaaaccccgtctctactaaatacaaaaaatagctgagcttggtggcgcacgcctatagtcctagctactggggaggctgaggtgggaggatcgcttgagcccaagaagtcgaggctgcagtgagccgagatcgcgccgctgcactccagcctgagcgacagggcgaggctctgtctcaaaacaaacaaacaaaaaaaaaaaggaaaggaaatataacacagtgaaatgaaaggattgagagaaatgaaaaatatacacgccacaaatgtgggagggcgataaccactcgtagaaagcgtgagaagttactacaagcggtcctcccgggcaccgtactgttccgctcccagaagccccgggcgccggaagtcgtcactcttaagaagggacggggccccacgctgcgcacccgcgggtttgctatggccatgagcagcggcggcagtggcggcggcgtgcccgagcaggaggattctgtgctgttccggagaggaacaggccagagcgatgactccgatatctgggacgacacagcccttatcaaggcctacgacaaggccgtggccagctttaagcacgccctgaagaatggcgatatctgcgagacaagcggaaagcctaagaccacccctaaaagaaagcccgccaagaaaaacaagtcccagaaaaaaaacaccgccgctagcctgcagcagtggaaggtgggcgacaaatgcagcgccatctggtccgaggacggctgcatctaccctgctaccatcgccagcatcgacttcaagcgggaaacctgcgtggtggtctacacaggctatggcaatagggaggaacaaaatctctctgatctgctgtctcctatttgtgaagtggctaacaacatcgagcagaacgcccaggaaaatgagaacgaaagccaagtgtccaccgacgagagcgagaacagcagaagccctggaaacaagtctgacaacatcaagcccaagtctgccccttggaacagcttcctgccccctcctcctccaatgcctggccccagactgggccccggcaagcctggcctgaagttcaacggccctcctccaccccctcctcctccacctccccatctgctgagctgctggctgcctccttttcccagcggcccccctatcatccccccaccacctcctatctgtcccgacagcctggacgacgccgatgctctgggatccatgctgatcagctggtacatgtctggctaccacaccggctactacatgggcttccggcagaaccagaaggaaggaagatgcagccacagcctgaactgagcggccacaaacaccattgtcacactccaacaaacaccattgtcacactccaacaaacaccattgtcacactccaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

(19) United States
(12) Patent Application Publication
(10) Pub. No.: US 2025/0011812 A1
(43) Pub. Date: Jan. 9, 2025

(54) GENE THERAPY FOR SPINAL MUSCULAR ATROPHY

(71) Applicant: University of Massachusetts, Boston, MA (US)

(72) Inventors: Jun Xie, Worcester, MA (US); Guangping Gao, Worcester, MA (US); Qing Xie, Worcester, MA (US); Hong Ma, Worcester, MA (US)

(73) Assignee: University of Massachusetts, Boston, MA (US)

(21) Appl. No.: 18/712,273

(22) PCT Filed: Nov. 22, 2022
(86) PCT No.: PCT/US2022/080315

§ 371 Date: May 22, 2024

Related U.S. Application Data
(60) Provisional application No. 63/341,650, filed on May 13, 2022.
(60) Provisional application No. 63/282,246, filed on Nov. 23, 2021.

(51) Int. Cl. C12N 15/86; A61K 48/00; A61P 25/00

(52) U.S. Cl. CPC C12N 15/86; A61P 25/00; A61K 48/00; C12N 2750/14122; C12N 2750/14143; C12N 2830/50

(57) ABSTRACT

Aspects of the disclosure relate to compositions and methods for treating spinal muscular atrophy (SMA). The disclosure is based, in part, on isolated nucleic acids and vectors (e.g., viral vectors, such as rAAV vectors) encoding SMN1. In some embodiments, the expression of SMN 1 is driven by a native SMN 1 promoter or a variant thereof. In some embodiments, isolated nucleic acids and vectors of the disclosure have reduced toxicity and/or increased transgene expression relative to previously described SMN-encoding vectors.

### RELATED APPLICATION

This application is a national stage filing under 35 U.S.C. § 371 of international PCT application PCT/US2022/080315, filed Nov. 22, 2022, which claims priority under 35 U.S.C. § 119(e) to U.S. provisional patent application, U.S. Ser. No. 63/341,650, filed May 13, 2022 and U.S. provisional patent application, U.S. Ser. No. 63/282,246, filed Nov. 23, 2021, the entire contents of each of which are incorporated by reference herein.

### REFERENCE TO AN ELECTRONIC SEQUENCE LISTING

The contents of the electronic sequence listing (U012070171US02-SEQ-KZM.xml; Size: 48,436 bytes; and Date of Creation: Apr. 25, 2024) is herein incorporated by reference in its entirety.

### BACKGROUND

Spinal muscular atrophy (SMA) is a neuromuscular disease caused by loss-of-function of survival of motor neuron 1 (SMN1) gene and characterized by the degeneration of motor neurons and progressive muscle weakness. SMA affects approximately 1 in 11,000 newborns and remains the leading genetic cause of infantile death. In the past decade, two major breakthroughs have been made to treat this devasting diseases by increasing SMN protein in human patients. The first is antisense oligonucleotides (ASOs), Nusinersen and risdiplam, which modify the splicing of existing SMN2 pre-mRNAs. The second is Zolgensma, a self-complementary adeno-associated virus 9 (scAAV9) mediated SMN1 gene replacement.

### SUMMARY

Aspects of the disclosure relate to compositions and methods for treating spinal muscular atrophy (SMA). The disclosure is based, in part, on isolated nucleic acids and vectors (e.g., viral vectors, such as rAAV vectors) encoding SMN1. In some embodiments, the expression of SMN1 is driven by a native SMN1 promoter or a variant thereof. In some embodiments, isolated nucleic acids and vectors of the disclosure have reduced toxicity and/or increased transgene expression relative to previously described SMN-encoding vectors.

Accordingly, in some aspects, the disclosure provides a recombinant adeno-associated virus (rAAV) vector comprising a transgene comprising an endogenous SMN1 promoter operably linked to a codon-optimized nucleic acid sequence encoding human SMN1, flanked by adeno-associated virus (AAV) inverted terminal repeats (ITRs).In some embodiments, a codon-optimized nucleic acid sequence comprises a nucleic acid sequence that is at least 70%, 80%, 90%, 95%, or 99% identical to the nucleic acid sequence set forth in SEQ ID NO: 1. In some embodiments, a codon-optimized nucleic acid sequence comprises or consists of the sequence set forth in SEQ ID NO: 1. In some embodiments, a codon-optimized nucleic acid sequence does not comprise the nucleic acid sequence set forth in SEQ ID NO: 2. In some embodiments, a human SMN1 comprises the amino acid sequence set forth in SEQ ID NO: 3.

In some embodiments, an endogenous SMN1 promoter is a human SMN1 promoter. In some embodiments, an endogenous SMN1 promoter comprises a nucleic acid sequence that is at least 70%, 80%, 90%, 95%, or 99% identical to the nucleic acid sequence set forth in SEQ ID NO: 4 or 5. In some embodiments, an endogenous SMN1 promoter comprises or consists of the nucleic acid sequence set forth in SEQ ID NO: 4. In some embodiments, an endogenous SMN1 promoter comprises or consists of the nucleic acid sequence set forth in SEQ ID NO: 5.

In some embodiments, the recombinant adeno-associated virus (rAAV) vector comprising a transgene comprising a promoter operably linked to a codon-optimized nucleic acid sequence encoding human SMN1, flanked by adeno-associated virus (AAV) inverted terminal repeats (ITRs), wherein the promoter is a constitutive promoter. In some embodiments, the promoter is a CB6 promoter. In some embodiments, the rAAV vector further comprising a CMV enhancer.

In some embodiments, the rAAV vector further comprising one or more miR-122 binding sites. In some embodiments, the one or more miR-122 binding sites are placed between the codon-optimized nucleic acid sequence encoding human SMN1 and the 3′ ITR. In some embodiments, the rAAV vector comprises a nucleic acid sequence at least 60%, at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9-11.

In some embodiments, at least one AAV ITR is an AAV2 ITR. In some embodiments, at least one AAV ITR is a mutant ITR (mTR).

In some aspects, the disclosure provides a vector comprising an rAAV vector as described herein. In some embodiments, a vector is a plasmid or a baculovirus vector.

In some aspects, the disclosure provides a cell comprising an rAAV vector as described herein or a vector as described herein. In some embodiments, the cell is a prokaryotic cell or a eukaryotic cell. In some embodiments, the cell is a mammalian cell, bacterial cell, or insect cell (e.g., SF9 cell).

In some aspects, the disclosure provides an isolated nucleic acid comprising the nucleic acid sequence set forth in any one of SEQ ID NO: 1, SEQ ID NO: 6, or SEQ ID NO: 7.

In some embodiments, an isolated nucleic acid further comprises an endogenous SMN1 promoter operably linked to the nucleic acid sequence. In some embodiments, an endogenous SMN1 promoter is a human SMN1 promoter. In some embodiments, an endogenous SMN1 promoter comprises or consists of the nucleic acid sequence set forth in SEQ ID NO: 4 or 5.

In some aspects, the disclosure provides a recombinant adeno-associated virus (rAAV) comprising a recombinant adeno-associated virus (rAAV) vector comprising a transgene comprising an endogenous SMN1 promoter operably linked to a codon-optimized nucleic acid sequence encoding human SMN1, flanked by adeno-associated virus (AAV) inverted terminal repeats (ITRs); and at least one AAV capsid protein.

In some embodiments, a codon-optimized nucleic acid sequence comprises a nucleic acid sequence that is at least 70%, 80%, 90%, 95%, or 99% identical to the nucleic acid sequence set forth in SEQ ID NO: 1. In some embodiments, a codon-optimized nucleic acid sequence comprises or consists of the sequence set forth in SEQ ID NO: 1. In some embodiments, a codon-optimized nucleic acid sequence does not comprise the nucleic acid sequence set forth in SEQ ID NO: 2. In some embodiments, a human SMN1 comprises the amino acid sequence set forth in SEQ ID NO: 3.

In some embodiments, an endogenous SMN1 promoter is a human SMN1 promoter. In some embodiments, an endogenous SMN1 promoter comprises a nucleic acid sequence that is at least 70%, 80%, 90%, 95%, or 99% identical to the nucleic acid sequence set forth in SEQ ID NO: 4 or 5. In some embodiments, an endogenous SMN1 promoter comprises or consists of the nucleic acid sequence set forth in SEQ ID NO: 4. In some embodiments, an endogenous SMN1 promoter comprises or consists of the nucleic acid sequence set forth in SEQ ID NO: 5.

In some embodiments, the recombinant adeno-associated virus (rAAV) vector comprising a transgene comprising a promoter operably linked to a codon-optimized nucleic acid sequence encoding human SMN1, flanked by adeno-associated virus (AAV) inverted terminal repeats (ITRs), wherein the promoter is a constitutive promoter. In some embodiments, the promoter is a CB6 promoter. In some embodiments, the rAAV vector further comprising a CMV enhancer.

In some embodiments, the rAAV vector further comprising one or more miR-122 binding sites. In some embodiments, the one or more miR-122 binding sites are placed between the codon-optimized nucleic acid sequence encoding human SMN1 and the 3′ ITR. In some embodiments, the rAAV vector comprises a nucleic acid sequence at least 60%, at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9-11. In some embodiments, at least one AAV ITR is an AAV2 ITR. In some embodiments, at least one AAV ITR is a mutant ITR (mTR). In some embodiments, an rAAV is a self-complementary AAV (scAAV).

In some embodiments, at least one AAV capsid protein is an AAV2, AAV3, AAV4, AAV5, AAV6, AAV7, AAV8, or AAV9 capsid protein, or a variant thereof. In some embodiments, at least one AAV capsid protein is an AAV9 capsid protein.

In some aspects, the present disclosure provides a recombinant adeno-associated virus (rAAV) comprising: (a) a self-complementary rAAV genome comprising: (i) a 5′ ITR; (ii) a human short SMN promoter comprising the nucleotide sequence of SEQ ID NO: 5; (iii) a codon optimized nucleic acid sequence encoding SMN1 as set forth in SEQ ID NO: 1; (iv) a poly A tail; and (v) a 3′ ITR; and (b) a AAV9 capsid protein. In some embodiments, the poly A tail is a rabbit globin poly A or a BGH poly A tail. In some embodiments, the rAAV further comprises one or more miR-122 binding sites.

In some aspects, the disclosure provides a pharmaceutical composition comprising an rAAV vector or rAAV as described herein, and a pharmaceutically acceptable excipient.

In some aspects, the disclosure provides a method for delivering a transgene to a cell, the method comprising administering an rAAV vector, rAAV, or pharmaceutical composition as described herein to a cell.

In some embodiments, a cell is a mammalian cell. In some embodiments, a cell is a human cell. In some embodiments, a cell is in a subject. In some embodiments, a subject has or is suspected of having spinal muscular atrophy (SMA).

In some aspects, the disclosure provides a method for preventing or treating spinal muscular atrophy (SMA) in a subject, the method comprising administering an rAAV vector, rAAV, or pharmaceutical composition as described herein to the subject.

In some embodiments, the subject is a mammal. In some embodiments, a subject is a human. In some embodiments, a subject has one or more mutations in a SMN1 gene.

In some embodiments, administering comprises systemic injection or local injection. In some embodiments, systemic injection comprises intravenous injection. In some embodiments, administering comprises injection to the central nervous system (CNS) of a subject.

In some embodiments, administration results in a reduced amount of dorsal root ganglion (DRG) toxicity in a subject relative to administration of a wild-type SMN1-encoding rAAV to the subject. In some embodiments, the administration results in a reduced amount of dorsal root ganglion (DRG) toxicity in the subject relative to administration of an rAAV comprising a constative promoter operably linked to a wild-type SMN1-coding sequence to the subject.

In some embodiments, the administration results in reduced liver toxicity in the subject relative to administration of an AAV vector comprising a constitutive promoter operably linked to a nucleic acid encoding human SMN1 to the subject.

In some embodiments, the administration results in reduced complication associated with SMA. In some embodiments, the complication of SMA comprises pulmonary infections, spinal deformities (e.g., scoliosis, hip subluxations/dislocations), joint contractures, or respiratory failure.

In some embodiments, the administration results in improved survival in the subject relative to administration of an AAV vector comprising a constitutive promoter operably linked to a nucleic acid encoding human SMN1 to the subject.

### BRIEF DESCRIPTION OF DRAWINGS

FIG. 1 shows a schematic representation of SMN1-encoding vectors of the disclosure. “scAAV-CB6-opt-SMN1” is a self-complementary AAV (scAAV) vector with a CB6 promoter and a codon-optimized SMN1 coding sequence. “SMN1p-SMN1” is a single-stranded AAV genome with a 2.0 kb endogenous human SMN1 promoter and a codon-optimized SMN1 coding sequence (CDS). “ssAAV-opt-SMN1 (MBL)” is a single-stranded AAV genome with a 2.0 kb endogenous human SMN1 promoter, a synthetic MBL intron, and a codon-optimized SMN1 coding sequence (CDS). “SMNsp-SMN1” is a self-complementary AAV vector with a 0.9 kb endogenous human SMN1 promoter and a codon-optimized SMN1 CDS. “Zolgensma” is a self-complementary AAV vector with a CMV enhancer/chicken beta-actin promoter and a wild-type human SMN1 CDS.

FIGS. 2A-2F show representative data for ssAAV9.opt-SMN1 and Zolgensma in SMNΔ7 mice. FIG. 2A shows a survival curve of SMNΔ7 mice treated with Zolgensma (2xE14 genome copies (GCs)/kg) or SMNp-SMN1 rAAV at three doses (2xE14 genome copies (GCs)/kg; 6.8xE13 GCs/kg, or 3.4xE13 GCs/kg); ssAAV9.opt-SMN1 treated at the same or ⅓ doses have better survival than mice treated with Zolgensma. FIGS. 2B and 2C show representative data indicating that ssAAV9.opt-SMN1 treated mice show similar body weight gain compared to Zolgensma treated mice. FIG. 2D shows a survival curve for scAAV9.opt-SMN1 and Zolgensma in SMNΔ7 mice; scAAV9.opt-SMN1 treated at the same or ⅓ doses have better survival than mice treated with Zolgensma. FIGS. 2E and 2F show representative data indicating that ssAAV9.opt-SMN1 treated mice show similar body weight gain compared to Zolgensma treated mice.

FIGS. 3A-3B show representative behavioral assay data for SMNΔ7 mice treated with scAAV9.opt-SMN1. FIG. 3A shows scAAV9.opt-SMN1 treated animals can right themselves from day 5 post-administration. FIG. 3B shows representative data indicating that all treated animals have functional muscle, as measured by rotarod test.

FIGS. 4A-4B show representative toxicology data. FIG. 4A shows histological data indicating that high levels of SMN1 overexpression are toxic to liver cells. Briefly, 5E+11 GC/mouse of AAV vectors were injected into animals at P0 via facial vein. Liver was collected at day 8 post injection. Liver damage was observed in AAVsc-CB6-PI-SMN1 (6/6 animals), and Zolgensma (4/7 animals) vector treated groups but not control empty AAV9 (0/8 animals) or ssAAV9-opt-SMN1 (0/8) vector treated groups. FIG. 4B shows representative alanine aminotransferase (ALT) assay test results.

FIG. 5 shows a schematic indicating proposed expression levels for SMN1-encoding vectors.

FIGS. 6A-6D show representative data for SMNΔ7 mice treated with Zolgensma, SMNp-SMN1 rAAV, or SMNsp-SMN1 rAAV. FIG. 6A shows a survival curve of SMNΔ7 mice treated with Zolgensma (5xE11 genome copies (GC)) or SMNp-SMN1 rAAV at two doses (5xE11 GC or 1.67xE11 GC). FIG. 6B shows body weight growth of SMNΔ7 mice treated with Zolgensma (5xE11 GC) or SMNp-SMN1 rAAV at two doses (5xE11 GC or 1.67xE11 GC). FIG. 6C shows a survival curve of SMNΔ7 mice treated with Zolgensma (5xE11 GC) or SMNsp-SMN1 rAAV at three doses (5xE11 GC, 1.67xE11 GC, or 0.8xE11 GC). FIG. 6D shows body weight growth of SMNΔ7 mice treated with Zolgensma (5xE11 GC) or SMNsp-SMN1 rAAV at two doses (5xE11 GC or 1.67xE11 GC). Untreated control animals are also shown in each figure.

FIGS. 7A-7D show that rAAV9sc-CMVen/CB-co-hSMN1 induced liver toxicity due to overexpression of hSMN1 in the liver. FIG. 7A shows that mice received rAAV9sc-CMVen/CB-co-hSMN1 treatment via facial vein injection showed yellow skin. FIG. 7B shows that liver toxicity was observed in mice received rAAV9sc-CMVen/CB-co-hSMN1 by liver histology. FIG. 7C shows the number of animals that had liver toxicity after rAAV9sc-CMVen/CB-co-hSMN treatment compared to a reference vector and untreated control (UC). FIG. 7D shows SMN1 expression in liver from mice treated with rAAV9sc-CMVen/CB-co-hSMN, the reference vector, or the untreated control.

FIGS. 8A-8C show that incorporating miR-122 binding site to CMVen/CB-co-hSMN vector abrogated SMN1 expression in the liver, thereby reducing liver toxicity. FIG. 8A shows the AAV vector with or without miR-122 binding sites. FIG. 8B shows that incorporation of miR-122 binding sites in CMVen/CB-co-hSMN vector (CB6-PI-opt hSMN1-miR-122 BS) abrogated SMN1 expression in the liver. FIG. 8C shows that mice treated with scAAV9-CB6-PI-opt hSMN1-miR-122 BS) had no liver toxicity compared to the mice treated with rAAV9sc-CMVen/CB-co-hSMN1.

FIGS. 9A-9L show that the 2^{nd} generation AAV (AAVsc-SMNp-co-hSMN1) resulted in improved therapeutic outcome in a SMA animal model. FIG. 9A shows that AAVsc-SMNp-co-hSMN1 resulted in higher expression efficiency in Neuro-2a cells compared to rAAV9sc-CMVen/CB6-co-hSMN1. FIG. 9B shows the experimental design of treating mice using AAVsc-SMNp-co-hSMN1 via facial vein injection, Mice were monitored daily after treatment. FIG. 9C shows that the mice treated with AAVsc-SMNsp-co-hSMN1 gained more body weight than the reference vector group. FIG. 9D shows that the administration with AAVsc-SMNsp-co-hSMN1 vector significantly improved the life span of the SMA mice. FIG. 9E shows that the AAVsc-SMNsp-co-hSMN1 treated SMA mice were healthy at 5 months old. FIG. 9F shows that the SMA mice treated with AAVsc-SMNsp-co-hSMN1 were able to righting themselves earlier than the reference ones. FIG. 9G are representative images from transverse abdominis (TVA) shows the treatment effect on the restoration of innervation in neuromuscular junctions (NMJs). FIG. 9H shows that the structures of neuromuscular junctions in the AAVsc-SMNsp-co-hSMN1-treated mice were restored close to that of wild-type mice, better than those of reference-treated SMA mice. FIG. 9I shows the difference stages of car necrosis as one of the complications in SMA mice. FIG. 9J shows that treatment with AAVsc-SMNsp-co-hSMN1 alleviated complication (e.g., car necrosis) in SMA mice. FIGS. 9K-9L show immunoblot assay indicating that the AAVsc-SMNsp-co-hSMN1 preferentially express in central nervous system other than peripheral tissues. The expression pattern of this vector is close to the nature distribution of SMN as in healthy control animals.

FIG. 10 shows a representative survival curve of SMA mice either not treated (NT), treated with a benchmark vector (BMK), or treated with scAAV9-SMNsp-hSMN1; scAAV9-SMNsp-hSMN1 treated have better survival than untreated mice, or mice treated with BMK.

### DETAILED DESCRIPTION

Aspects of the disclosure relate to compositions and methods for treating spinal muscular atrophy (SMA). The disclosure is based, in part, on isolated nucleic acids and vectors (e.g., viral vectors, such as rAAV vectors) encoding SMN1. In some embodiments, the expression of SMN1 is driven by a native SMN1 promoter or a variant thereof. In some embodiments, isolated nucleic acids and vectors of the disclosure have reduced toxicity and/or increased transgene expression and/or reduced off-target tissue expression, relative to previously described SMN-encoding vectors.

### Survival of Motor Neuron 1 (SMN1)

Aspects of the disclosure relate to compositions (e.g., isolated nucleic acids, vectors such as rAAV vectors, rAAVs, etc.) that encode a survival of motor neuron 1 (SMN1) protein. SMN1 protein is a component of a complex that catalyzes the assembly of small nuclear ribonucleoproteins (snRNPs) and plays an important role in the splicing of cellular pre-mRNAs. Mutations in SMN1 and SMN2 are associated with a Spinal Muscular Atrophy (SMA), a severe neuromuscular disorder that results in the loss of motor neurons and progressive muscle wasting. Spinal muscular atrophy (SMA) is a rare neuromuscular disorder that results in the loss of motor neurons and progressive muscle wasting. It is usually diagnosed in infancy or early childhood and if left untreated it is the most common genetic cause of infant death. It may also appear later in life and then have a milder course of the disease. The common feature is progressive weakness of voluntary muscles, with arm, leg and respiratory muscles being affected first. Patients with SMA could also develop complications associated problems may include poor head control, difficulties swallowing, pulmonary infections, spinal deformities (e.g., scoliosis, hip subluxations/dislocations), joint contractures, and respiratory failure.

The age of onset and the severity of symptoms form the basis of the traditional classification of spinal muscular atrophy into a number of types.

Spinal muscular atrophy is due to an abnormality (mutation) in the SMN1 gene which encodes SMN, a protein necessary for survival of motor neurons. Loss of these neurons in the spinal cord prevents signaling between the brain and skeletal muscles. Another gene, SMN2, is considered a disease modifying gene, since usually the more the SMN2 copies, the milder is the disease course. The diagnosis of SMA is based on symptoms and confirmed by genetic testing.

Outcomes in the natural course of the disease vary from death within a few weeks after birth in the most acute cases to normal life expectancy in the protracted SMA forms.

Medications that target the genetic cause of the disease include nusinersen, risdiplam, and the gene therapy medication onasemnogene abeparvovec. Supportive care includes physical therapy, occupational therapy, respiratory support, nutritional support, orthopaedic interventions, and mobility support.

In humans, SMN1 is encoded by a SMN1 gene, for example as set forth in NCBI Reference Sequence Nos. NM_000344, NM_001297715, and NM_022874. In some embodiments, a SMN1 protein comprises the amino acid sequence set forth in any one of NCBI Reference Sequence Nos. NP_059107, NP_075013, NP_075014, NP_075015, NP_000335. In some embodiments, a SMN1 protein comprises the amino acid sequence set forth in SEQ ID NO: 3. In some embodiments, a SMN1 protein comprises an amino acid sequence that is at least 50%, 60%, 70%, 75%, 80%, 90%, 95%, or 99% identical to the amino acid sequence set forth in SEQ ID NO: 3.

In some aspects, the disclosure relates to isolated nucleic acids comprising a codon-optimized (e.g., optimized to increase expression in human cells, reduce toxicity, reduce immunogenicity, etc.) nucleic acid sequence that encodes a SMN1 protein. In some embodiments, an isolated nucleic acid encoding a SMN1 protein comprises a nucleic acid sequence that is at least 70%, 75%, 80%, 90%, 95%, or 99% identical to the nucleic acid sequence set forth in SEQ ID NO: 1. In some embodiments, an isolated nucleic acid encoding a SMN1 protein comprises the nucleic acid sequence set forth in SEQ ID NO: 1. In some embodiments, an isolated nucleic acid encoding a SMN1 protein consists of the nucleic acid sequence set forth in SEQ ID NO: 1.

In some embodiments, an isolated nucleic acid encoding a SMN1 protein comprises at least one (e.g., 1, 2, 3, 4, 5, 10, 15, 20, 25, 50, 100, 150, 200, 250, or more) nucleotide substitutions, insertions, deletions, or any combination thereof, relative to a wild-type SMN1-encoding nucleic acid sequence, for example the nucleic acid sequence set forth in SEQ ID NO: 2.

A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are AAV2 ITRs. In some embodiments, at least one AAV ITR is a truncated AAV ITR (e.g., a mutant ITR, also referred to as an mTR), for example a ΔITR as described, for example by McCarty (2008) Molecular Therapy 16(10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein.

A region comprising a transgene (e.g., a transgene encoding a SMN1 protein, etc.) may be positioned at any suitable location of the isolated nucleic acid that will enable expression of the at least one transgene, the selectable marker protein, or reporter protein.

It should be appreciated that in cases where a transgene encodes more than one gene product (e.g., a SMN1 protein and another protein or interfering nucleic acid), each gene product may be positioned in any suitable location within the transgene. For example, a nucleic acid encoding a first polypeptide may be positioned in an intron of the transgene and a nucleic acid sequence encoding a second polypeptide may be positioned in another untranslated region (e.g., between the last codon of a protein coding sequence and the first base of the poly-A signal of the transgene).

A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the disclosure may also contain an intron, desirably located between the promoter/enhancer sequence and the transgene. One possible intron sequence is derived from SV-40, and is referred to as the SV-40 T intron sequence. In some embodiments, an intron is a non-native intron or synthetic intron (e.g., a MBL intron). Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional, and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an RNA pol II promoter. In some embodiments, a promoter is an RNA pol III promoter, such as U6 or H1. In some embodiments, a promoter is an RNA pol II promoter. In some embodiments, a promoter is a chicken β-actin (CBA) promoter. In some embodiments, a promoter comprises a U1a promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

Aspects of the disclosure relate to isolated nucleic acids and rAAV vectors comprising a nucleic acid sequence encoding SMN1 (e.g., a codon-optimized nucleic acid sequence encoding SMN1) operably linked to a native promoter. In some embodiments a native promoter comprises a human SMN1 promoter, or a variant thereof. In some embodiments, a human SMN1 promoter comprises the nucleic acid sequence set forth in SEQ ID NO: 4. In some embodiments, a promoter comprises a portion of a human SMN1 promoter which comprises the nucleic acid sequence set forth in SEQ ID NO: 5 (i.e., short hSMN1 promoter). In some embodiments, a human SMN1 promoter or variant thereof comprises a nucleic acid sequence that is at least 50%, at least 60%, at least 70%, at least 80%, at least 90%, at least 91%, at least 92%, at least 93%, at least 94%, at least 95%, at least 96%, at least 97%, at least 98%, at least 99%, or 100% identical to the nucleic acid sequence set forth in SEQ ID NO: 4 or SEQ ID NO: 5. Human SMN1 promoters are generally known in the art, for example as described by Echaniz-Laguna et al., Am. J. Hum. Genet. 64:1365-1370, 1999. The native promoter may be preferred when it is desired that expression of the transgene should mimic the native expression (e.g., express physiological levels of hSMN1 expression in the appropriate cell types). The native promoter may be used when expression of the transgene must be regulated temporally or developmentally, or in a tissue-specific manner, or in response to specific transcriptional stimuli. Without wishing to be bound by any theory, use of a human SMN1 promoter in isolated nucleic acids and rAAV vectors described herein regulates expression of human SMN1 protein from the vectors, and reduces toxicity, for example dorsal root ganglion (DRG) toxicity or hepatotoxicity, in a subject relative to expression of human SMN1 protein from isolated nucleic acids and rAAV vectors comprising other promoters, for example CMV promoter, chicken-beta actin (CBA) promoter, CB6 promoter, etc. In a further embodiment, other native expression control elements, such as enhancer elements, polyadenylation sites, and/or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, the promoter preferentially drives transgene expression in certain tissues. In some embodiments, the disclosure provides a nucleic acid comprising a tissue-specific promoter operably linked to a transgene. As used herein, “tissue-specific promoter” refers to a promoter that preferentially regulates (e.g., drives or up-regulates) gene expression in a particular cell type relative to other cell types. A cell-type-specific promoter can be specific for any cell type, such as central nervous system (CNS) cells, liver cells (e.g., hepatocytes), heart cells, muscle cells, etc. In some embodiments, a tissue-specific promoter is a muscle tissue or cell-specific promoter. Examples of CNS-specific promoters include but are not limited to synapsin (Syn), GFAP, Ca 2⁺/calmodulin-dependent protein kinase II (hCAMKII), etc.

In some aspects, the disclosure relates to isolated nucleic acids comprising a transgene encoding one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding one or more complement control proteins as described herein, and a poly A sequence.

In some embodiments, the rAAV vector described herein comprises a nucleic acid sequence at least 60%, at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6-8.

In some embodiments, transgene expression causes overexpression of the transgene in the liver, resulting in liver toxicity (see, e.g., Hinderer et al., Severe Toxicity in Nonhuman Primates and Piglets Following High-Dose Intravenous Administration of an Adeno-Associated Virus Vector Expressing Human SMN, Volume: 29 Issue 3, 285-298: Mar. 1, 2018. In some embodiments, in order to reduce liver toxicity, the AAV vector comprises a transgene comprises one or more (e.g., 1, 2, 3, 4, 5, or more) miRNA binding sites that de-target expression of a transgene from liver cells. For example, in some embodiments, a transgene comprises one or more miR-122 binding sites. In some embodiments, the rAAV vectors described herein comprises one or more miR-122 binding sites. In some embodiments, the rAAV vectors comprising miR-122 binding sites comprises a nucleic acid sequence at least 60%, at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9-11.

In some embodiments, a transgene comprises one or more (e.g., 1, 2, 3, 4, 5, or more) miRNA binding sites that de-target expression of a transgene from immune cells (e.g., antigen presenting cells (APCs), such as macrophages, dendrites, etc.). Incorporation of miRNA binding sites for immune-associated miRNAs may de-target transgene (e.g., one or more inhibitory nucleic acids) expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is an miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδ T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152. In some embodiments, a transgene described herein comprises one or more binding sites for miR-142.

### Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., muscle tissues, ocular tissues, neurons, etc.). The AAV capsid is an important element in determining these tissue-specific targeting capabilities (e.g., tissue tropism). Thus, an rAAV having a capsid appropriate for the tissue being targeted can be selected.

In some embodiments, rAAVs of the disclosure comprise a nucleotide sequence as set forth in SEQ ID NO: 1, 6 or 7, or encode a protein having an amino acid sequence as set forth in SEQ ID NO: 3. In some embodiments, rAAVs of the disclosure comprise a nucleotide sequence that is 99% identical, 95% identical, 90% identical, 85% identical, 80% identical, 75% identical, 70% identical, 65% identical, 60% identical, 55% identical, or 50% identical to a nucleotide sequence as set forth in SEQ ID NO: 1, 6-11.

In some aspects, the present disclosure provides a recombinant adeno-associated virus (rAAV) comprising: (a) a self-complementary rAAV genome comprising: (i) a 5′ ITR; (ii) a human short SMN promoter comprising the nucleotide sequence of SEQ ID NO: 5; (iii) a codon optimized nucleic acid sequence encoding SMN1 as set forth in SEQ ID NO: 1; (iv) a poly A tail; and (v) a 3′ ITR; and (b) a AAV9 capsid protein. In some embodiments, the poly A tail is a rabbit globin poly A or a BGH poly A tail. In some embodiments, the rAAV further comprises one or more miR-122 binding sites.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for central nervous system (CNS) tissues. In some embodiments, an AAV capsid protein targets neuronal cell types, astrocytes, oligodendrocytes, glial cells, etc. In some embodiments, an AAV capsid protein is of an AAV serotype selected from the group consisting of AAV1, AAV2, AAV3, AAV4, AAV5, AAV6, AAV7, AAV8, AAVrh8, AAV9, AAV10, AAVrh10, AAV.PHP-CB, AAVrh39, AAVrh43, and variants of any of the foregoing. In some embodiments, the rAAV comprises an AAV9 capsid protein.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector (scAAV or scrAAV vector), for example as described by McCarthy (2008) Molecular Therapy 16(10):1648-1656.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a codon-optimized coding sequence encoding a transgene (e.g., SMN1). A “host cell” refers to any cell that harbors, or is capable of harboring, a substance of interest. Often a host cell is a mammalian cell. In some embodiments, a host cell is a neuron. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a central nervous system cell, for example a neuron or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, etc. Thus, the term includes cloning and expression vehicles, as well as viral vectors. In some embodiments a vector comprises a baculovirus vector, which are useful for producing viral particles in certain insect cells (e.g., SF9 cells). In some embodiments, useful vectors are contemplated to be those vectors in which the nucleic acid segment to be transcribed is positioned under the transcriptional control of a promoter.

### Delivery of a Transgene to Tissue

The isolated nucleic acids, rAAVs, and compositions of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, a subject is human.

Delivery of the rAAVs may be by, for example intramuscular injection or infusion into the muscle tissue or cells of a subject. As used herein, “muscle tissues” refers to any tissue derived from or contained in skeletal muscle, smooth muscle, or cardiac muscle of a subject. Non-limiting examples of muscle tissues include skeletal muscle, smooth muscle, cardiac muscle, myocytes, sarcomeres, myofibrils, etc.

Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

Aspects of the instant disclosure relate to compositions comprising a recombinant AAV comprising a capsid protein and a nucleic acid encoding a transgene, wherein the transgene comprises a codon-optimized nucleic acid sequence encoding a SMN1 protein. In some embodiments, the nucleic acid further comprises AAV ITRs. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), intraocular injection, subretinal injection, oral, inhalation (including intranasal and intratracheal delivery), intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art. In some embodiments, an rAAV as described herein is administered to a subject in a dose ranging between about 1 ml to about 100 ml of solution containing from about 10⁹ to 10¹⁶ genome copies. In some cases, a dosage between about 10¹¹ to 10¹³ rAAV genome copies is administered.

An effective amount of an rAAV is an amount sufficient to target infect an animal, target a desired tissue. In some embodiments, an effective amount of an rAAV is administered to the subject during a pre-symptomatic stage of degenerative disease. In some embodiments, a subject is administered an rAAV or composition after exhibiting one or more signs or symptoms of degenerative disease.

An effective amount of an rAAV may also depend on the mode of administration. For example, targeting a muscle tissue (e.g., muscle cells) by intramuscular administration or subcutaneous injection may require different (e.g., higher or lower) doses, in some cases, than targeting muscle tissue by another method (e.g., systemic administration, topical administration, etc.). In some embodiments, intramuscular injection (IM) of rAAV having certain serotypes (e.g., AAV2, AAV6, AAV9, etc.) mediates efficient transduction of muscle cells. Thus, in some embodiments, the injection is intramuscular injection (IM). In some embodiments, the injection is systemic administration (e.g., intravenous injection). In some cases, multiple doses of a rAAV are administered. In some embodiments, the administration is systemic administration. In some embodiments, the systemic administration comprises intravenous administration. In some embodiments, the administration is local administration to the central nervous system, for example by intracerebral injection, intrathecal injection, intracranial injection, etc.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well known in the art and, include, for example, addition of surfactants, pH adjustment, salt concentration adjustment, etc. (See, e.g., Wright FR, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intraocularly, subretinally, subcutaneously, intraopancreatically, intranasally, parenterally, intravenously, intramuscularly, intrathecally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringeability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Therapeutic Methods

Aspects of the disclosure relate to compositions and methods for preventing or treating certain neurological and/or neuromuscular diseases, for example Spinal Muscular Atrophy (SMA). The disclosure is based, in part, on isolated nucleic acids, rAAVs, etc., comprising a codon-optimized nucleic acid sequence which encode a SMN1 protein. In some embodiments, the rAAV vector comprises a native (e.g., human) SMN/promoter, for example as set forth in SEQ ID NO: 4 or 5.

The disclosure is based, in part, on increasing SMN1 protein expression using rAAVs and vectors described herein, which also result in reduced toxicity in a subject (e.g., relative to a subject that has been administered previously used rAAV-SMN1 vectors, e.g., Zolgensma). In some embodiments, the reduced toxicity is reduced hepatotoxicity. In some embodiments, the reduced toxicity is reduced dorsal root ganglion (DRG) toxicity. The amount of reduction in toxicity in a subject may vary. In some embodiments toxicity is reduced between 2-fold and 5-fold, 3-fold and 10-fold, 5-fold and 20-fold, 10-fold and 50-fold, 30-fold and 80-fold, or 50-fold to 100-fold (e.g., relative to a subject that has been administered previously used rAAV-SMN1 vectors). In some embodiments, toxicity is reduced more than 100-fold (e.g., relative to a subject that has been administered previously used rAAV-SMN1 vectors, e.g., Zolgensma). Methods of measuring toxicity in a subject are known, and include measurement of cytotoxicity (e.g., cell death), measurement of hepatotoxicity (e.g., by ALT assay), etc.

In some embodiments, the administration results in reduced complication associated with SMA. In some embodiments, the complication of SMA comprises pulmonary infections, spinal deformities (e.g., scoliosis, hip subluxations/dislocations), joint contractures, or respiratory failure.

In some embodiments, the administration results in improved survival in the subject relative to administration of an AAV vector comprising a constitutive promoter operably linked to a nucleic acid encoding human SMN1 to the subject. In some embodiments, survival of the subject is increased by at least 10%, at least 20%, at least 30%, at least 40%, at least 50%, at least 60%, at least 70%, at least 80%, at least 90%, or 100% relative to a subject that has been administered previously used rAAV-SMN1 vectors, e.g., Zolgensma. Any suitable known methods of measuring survival rate can be used by the present disclosure.

In some embodiments, a subject is a mammalian subject, for example a human subject. In some embodiments, a subject is characterized as having one or more mutations in a SMN1 or SMN2 gene, for example one or more mutations resulting in reduced (or absence) of functional SMN1 protein in the cells of the subject. Examples of SMN1 mutations are described, for example by Wirth, Hum Mutat. 2000; 15(3):228-37. In some embodiments, a subject has reduced (or no) functional SMN1 protein in their cells. In some embodiments, the subject is a non-human mammal, for example, mouse, rat, goat, sheep, pig, cow, camel, llama, or non-human primates.

In some embodiments, administering the isolated nucleic acids, the rAAVs, or the compositions described herein to a cell or subject increases SMN1 expression in the cell or subject by between 2-fold and 100-fold (e.g., 2-fold, 5-fold, 10-fold, 20-fold, 50-fold, 75-fold, 100-fold, etc.) compared to a control subject. As used herein a “control” subject refers to a subject that is not administered the isolated nucleic acids, the rAAVs, or the compositions described herein. In some embodiments, a control subject is the same subject that is administered the isolated nucleic acids, the rAAVs, or the compositions described herein (e.g., prior to the administration).

In some aspects, the disclosure relates to a method for treating SMA in a subject, the method comprising: administering to the subject an isolated nucleic acid, rAAV, or pharmaceutical composition as described herein.

As used herein, the term “preventing” refers to the application or administration of a composition comprising a transgene encoding a SMN1 protein to a subject, who has not had a symptom, or a disease associated with aberrant SMN1 activity but at a predisposition toward a disease associated with aberrant SMN1 activity, with the purpose to stop or slow down the development of one or more symptoms of the disease associated with aberrant SMN1 activity. In some embodiments, the disease is SMA. In some embodiments, “preventing” also refers to stop or slow down the development of a complication associated with aberrant SMN1 activity (e.g., SMA) as described herein.

As used herein, the term “treating” refers to the application or administration of a composition comprising a transgene encoding a SMN1 protein to a subject, who has a symptom or a disease associated with aberrant SMN1 activity, or a predisposition toward a disease associated with aberrant SMN1 activity, with the purpose to cure, heal, alleviate, relieve, alter, remedy, ameliorate, improve, or affect the disorder, the symptom of the disease, or the predisposition toward a disease associated with aberrant SMN1 activity. In some embodiments, the disease is SMA. In some embodiments, “treating” also refers to cure, heal, alleviate, relieve, alter, remedy, ameliorate, improve, or affect a complication associated with aberrant SMN1 activity (e.g., SMA) as described herein.

Alleviating a disease associated with aberrant SMN1 activity includes delaying the development or progression of the disease, or reducing disease severity. Alleviating the disease does not necessarily require curative results. As used therein, “delaying” the development of a disease (such as a disease associated with aberrant SMN1 activity, such as SMA) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with aberrant SMN1 activity or angiogenesis includes initial onset and/or recurrence.

Compositions described herein may be administered (or a transgene expressed) in one or more target cells of a subject (e.g., a mammalian subject, such as a human). In some embodiments, a composition (e.g., isolated nucleic acid, rAAV, etc.) is administered to a cell of the central nervous system (CNS) of a subject. Examples of CNS cells include but are not limited to neurons, astrocytes, glial cells, etc.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or intravenous needle tubing and bag, particularly in the case of the kits for producing specific somatic animal models.

The kit may have a variety of forms, such as a blister pouch, a shrink wrapped pouch, a vacuum scalable pouch, a scalable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for constructing an AAV vector as described herein. In addition, kits of the disclosure may include, instructions, a negative and/or positive control, containers, diluents and buffers for the sample, sample preparation tubes and a printed or electronic table of reference AAV sequence for sequence comparisons.

### EXAMPLE

### Example 1

Intravenous administration of Zolgensma in individuals under 2 years of age has shown significant clinical benefits in type 1 SMA infants but an acute elevation in liver transaminases was observed in most patients. Systemic administration of an AAV9-like SMN vector has been observed to induce toxicity in large animals, including dorsal root ganglia (DRG) neurodegeneration and inflammation. Long-term AAV9-mediated SMN overexpression in mouse models has been observed to induce dose-dependent, late-onset motor dysfunction. Altogether, the SMN1 expression beyond physiological levels delivered by AAV may be a safety concern for AAV-based SMA gene therapy.

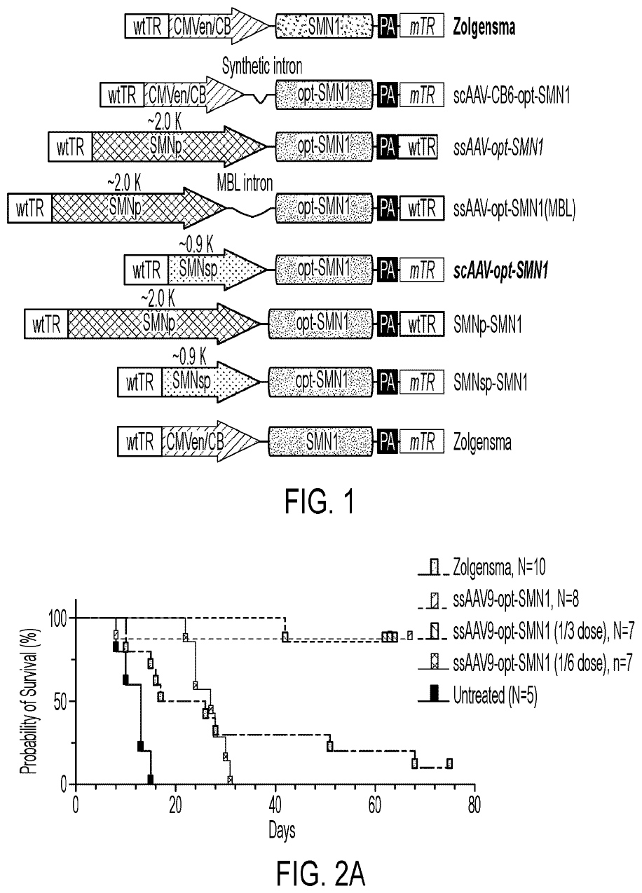

This example describes engineering of AAV-SMN1 vectors to address potential safety concerns. First, the SMN1 coding sequence (CDS) has been codon-optimized to maximize SMN1 expression in order to reduce vector doses delivered to patients. Second, an endogenous SMN1 promoter was used to drive AAV-based SMN1 expression in patients for an increased safety profile. FIG. 1 shows a schematic representation of SMN1-encoding vectors of the disclosure. “scAAV-CB6-opt-SMN1” is a self-complementary AAV (scAAV) vector with a CB6 promoter and a codon-optimized SMN1 coding sequence. “SMNp-SMN1” is a single-stranded AAV genome with a 2.0 kb endogenous human SMN1 promoter and a codon-optimized SMN1 coding sequence (CDS). “ssAAV-opt-SMN1 (MBL)” is a single-stranded AAV genome with a 2.0 kb endogenous human SMN1 promoter, a synthetic MBL intron, and a codon-optimized SMN1 coding sequence (CDS). “SMNsp-SMN1” is a self-complementary AAV vector with a 0.9 kb endogenous human SMN1 promoter and a codon-optimized SMN1 CDS. “Zolgensma” is a self-complementary AAV vector with a CMV enhancer/chicken beta-actin promoter and a wild-type human SMN1 CDS.

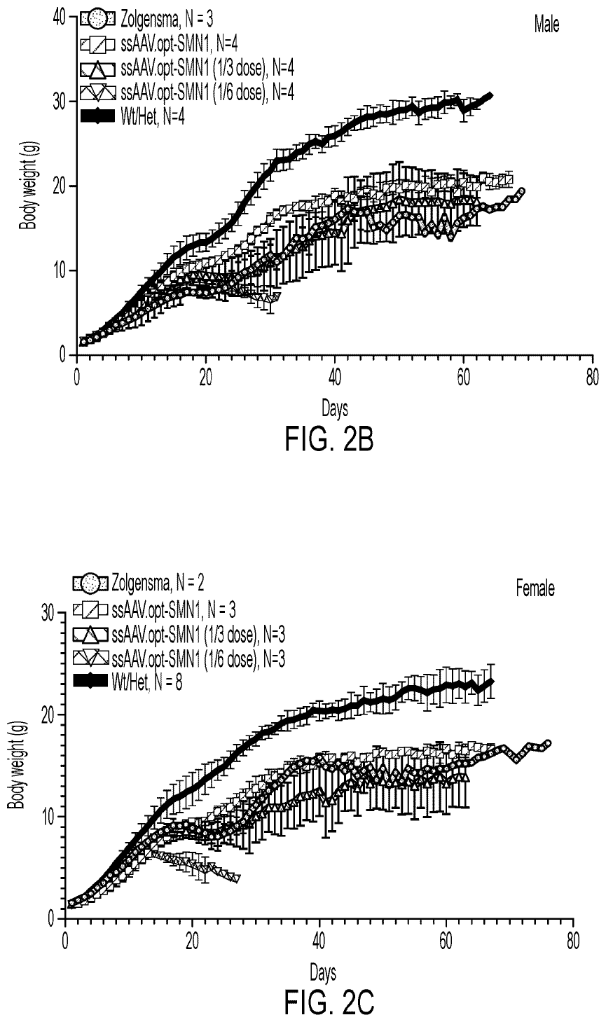

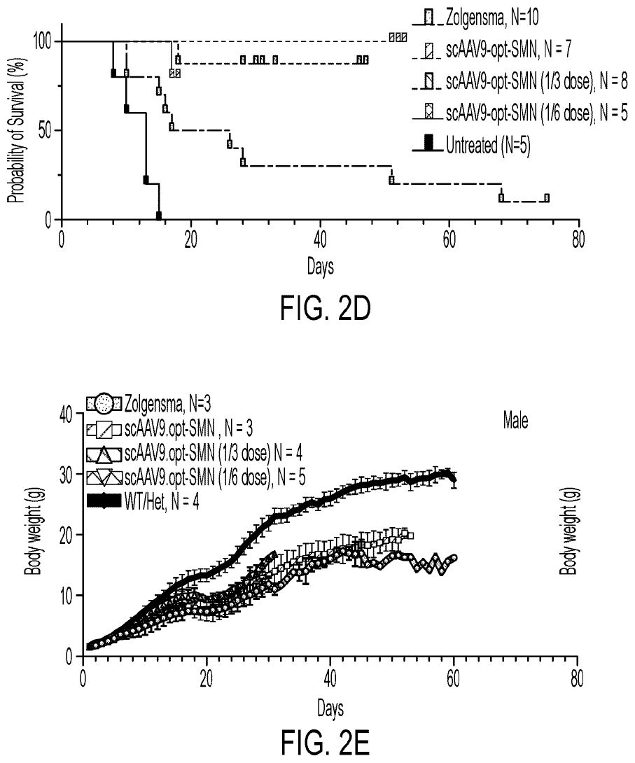

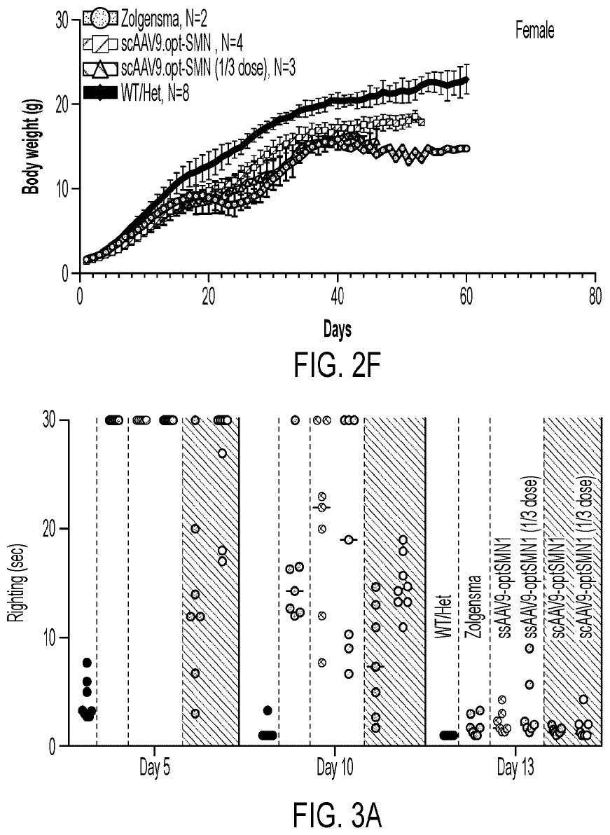

Expression of SMN1-encoding rAAV vectors was investigated in vivo. FIGS. 2A-2F show representative data for ssAAV9.opt-SMN1 and Zolgensma in SMNΔ7 mice. FIG. 2A shows a survival curve of SMNΔ7 mice treated with Zolgensma (2xE14 genome copies (GCs)/kg) or SMNp-SMN1 rAAV at three doses (2xE14 genome copies (GCs)/kg; 6.8xE13 GCs/kg, or 3.4xE13 GCs/kg); ssAAV9.opt-SMN1 treated at the same or ⅓ doses have better survival than mice treated with Zolgensma. FIGS. 2B and 2C show representative data indicating that ssAAV9.opt-SMN1 treated mice show similar body weight gain compared to Zolgensma treated mice. FIG. 2D shows a survival curve for scAAV9.opt-SMN1 and Zolgensma in SMNΔ7 mice; scAAV9.opt-SMN1 treated at the same or 1/3 doses have better survival than mice treated with Zolgensma. FIGS. 2E and 2F show representative data indicating that ssAAV9.opt-SMN1 treated mice show similar body weight gain compared to Zolgensma treated mice.

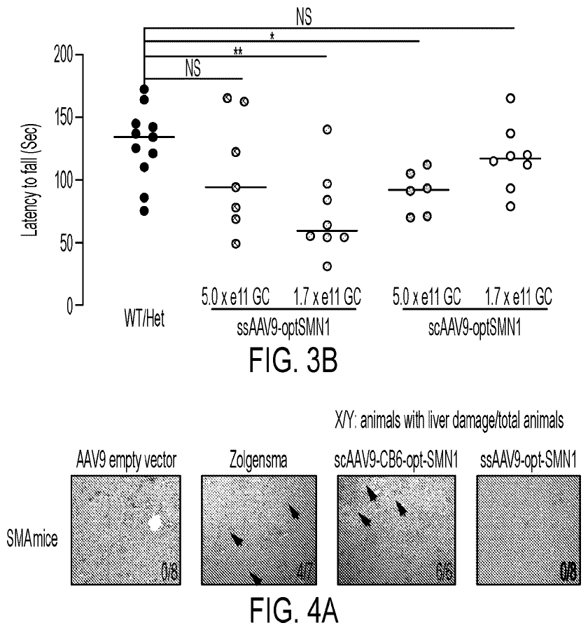

FIGS. 3A-3B show representative behavioral assay data for SMNΔ7 mice treated with scAAV9.opt-SMN1. FIG. 3A shows scAAV9.opt-SMN1 treated animals can right themselves from day 5 post-administration. FIG. 3B shows representative data indicating that all treated animals have functional muscle, as measured by rotarod test.

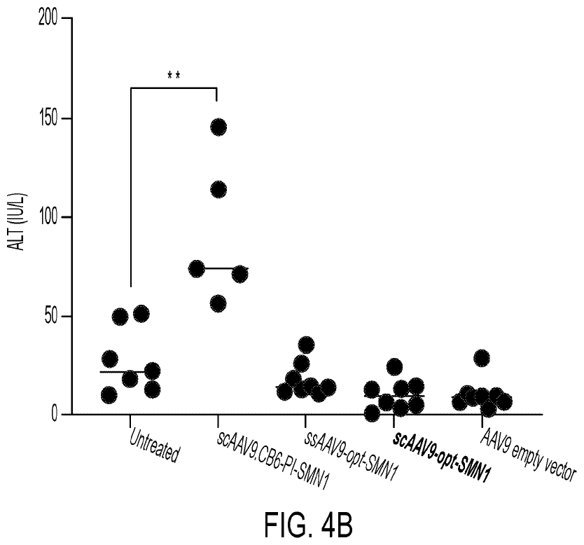

FIGS. 4A-4B show representative toxicology data. FIG. 4A shows histological data indicating that high levels of SMN1 overexpression are toxic to liver cells. Briefly, 5E+11 GC/mouse of AAV vectors were injected into animals at P0 via facial vein. Liver was collected at day 8 post injection. Liver damage was observed in AAVsc-CB6-PI-SMN1 (6/6 animals), and Zolgensma (4/7 animals) vector treated groups but not control empty AAV9 (0/8 animals) or ssAAV9-opt-SMN1 (0/8) vector treated groups. FIG. 4B shows representative alanine aminotransferase (ALT) assay test results.

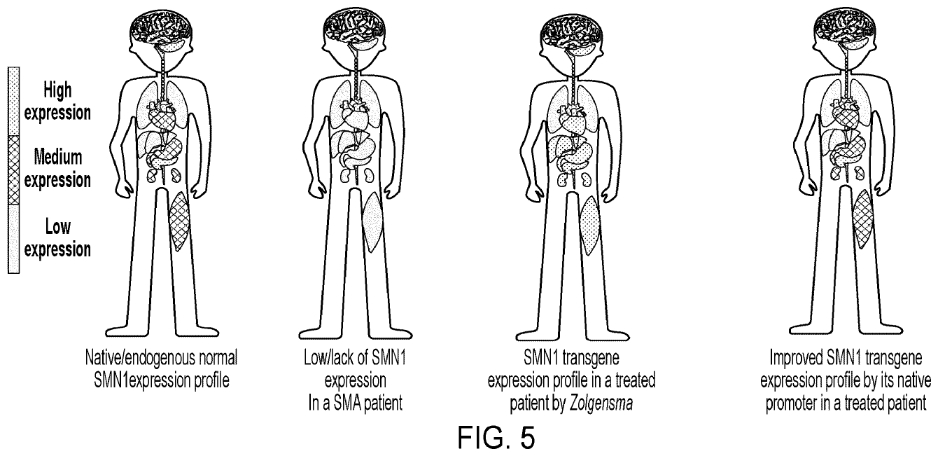

FIG. 5 shows a schematic indicating proposed expression levels for SMN1-encoding vectors. Briefly, SMN1-encoding vectors described by the disclose allow for high levels of expression of SMN1 in central nervous system (CNS) tissue without overexpression in muscle or other non-target organs, which may cause hepatotoxicity in a subject.

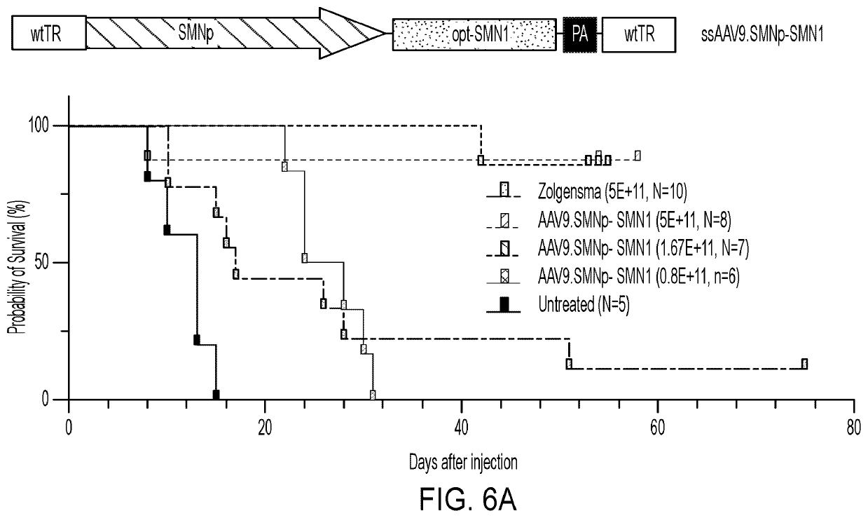

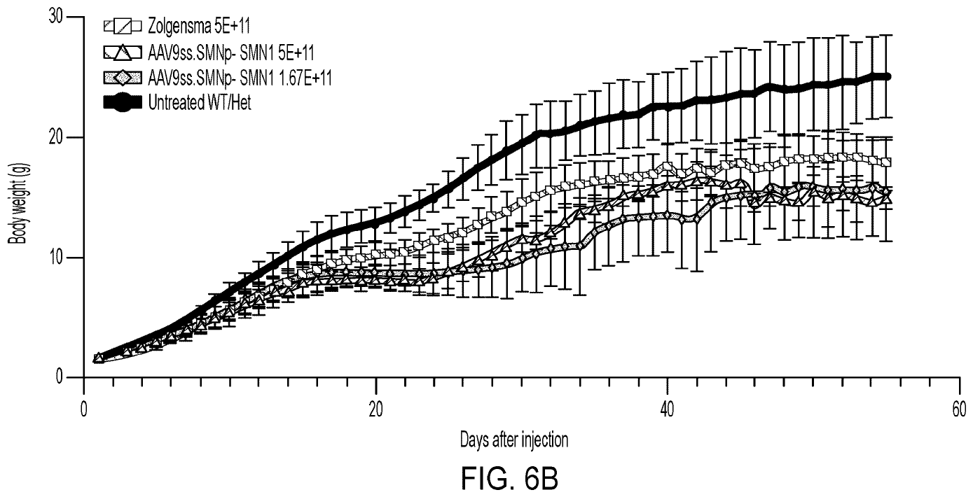

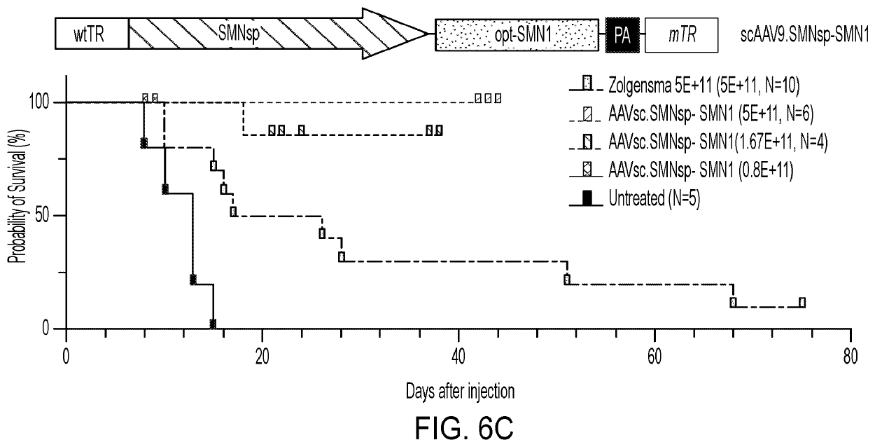

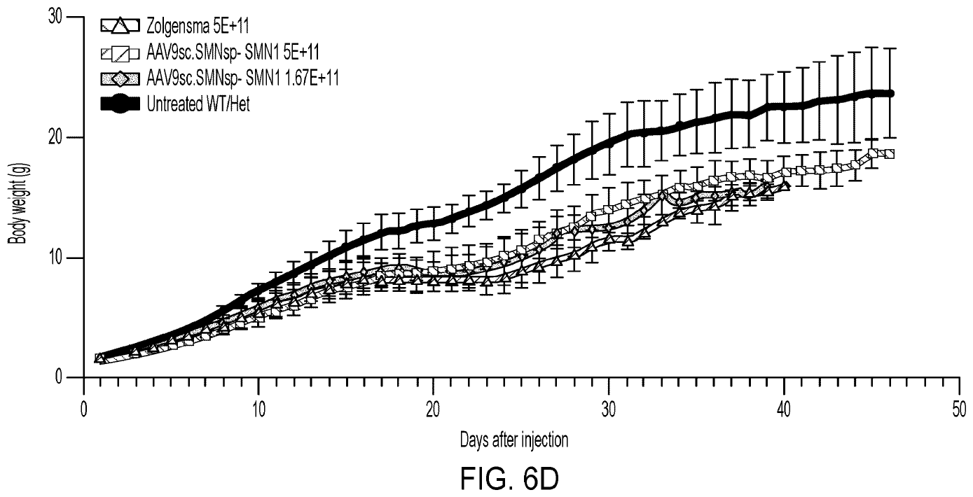

The three improved SMN1-encoding rAAV vectors described in FIG. 1 were packaged into AAV9 capsids. The rAAVs were then injected into SMNΔ7 mice at P1, and the mice were monitored for body weight and survival. Both of the engineered AAV-SMN1 vectors (e.g., SMNp-SMN1 and SMNsp-SMN1) outperform Zolgensma in survival. A one third-dose (relative to Zolgensma) of the engineered vectors (e.g., 1.67xE11 genome copies (GC) per mouse) was observed to extend the lifespan more efficiently than Zolgensma at 5xE11 GC/per mouse (FIGS. 6A and 6C). FIG. 6B shows that SMNΔ7 mice treated with 5xE11 GC of SMNp-SMN1 gained more weight than mice treated with Zolgensma after day 17, and the dose of 1.67xE11 GC of SMNp-SMN1 achieved similar weight at day 55 compared to the Zolgensma group. The SMNsp-SMN1 vector treated mice at two different doses (5xE11 GC and 1.67xE11 GC) also showed growth superiority from ˜day 23 compared to the Zolgensma group at the dose of 5xE11 GC (FIG. 6D).

### Example 2

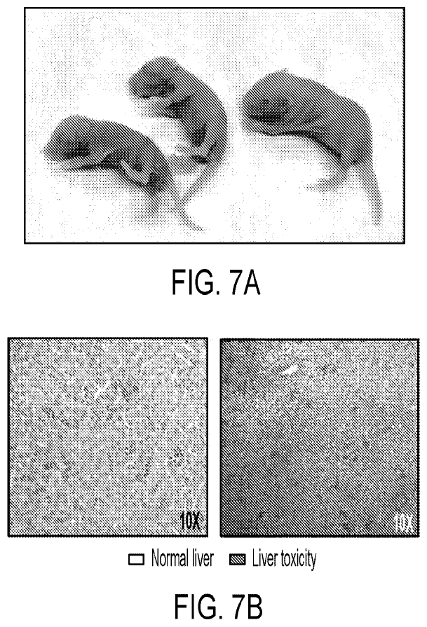

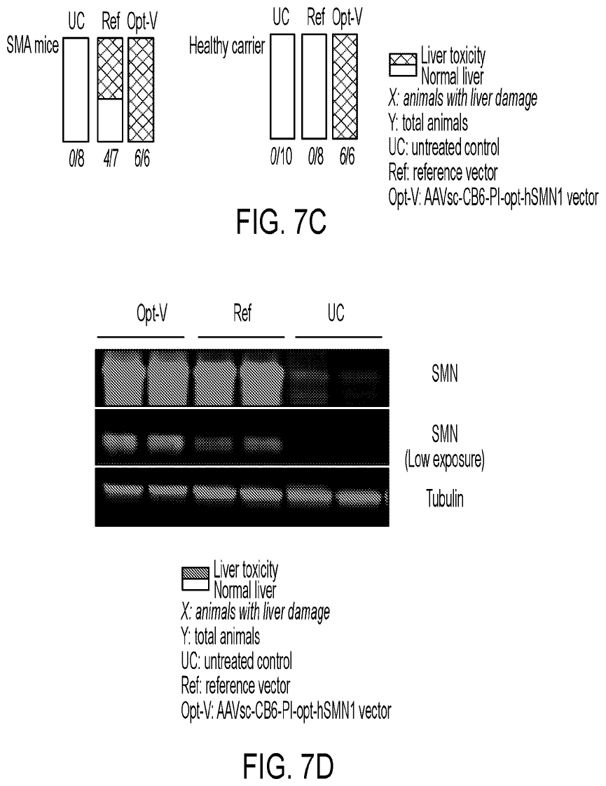

To test whether an AAV vector with codon optimized sequence of hSMN1 (co-hSMN1) under CMVen/CB promoter (CMVen/CB-co-hSMN1; SEQ ID NO: 8) would induce liver toxicity, the AAV vector was packaged into AAV9 capsid (rAAV9sc-CMVen/CB-co-hSMN1) and the rAAV was administered to postnatal day 1 SMNdelta7 mice at 3.3×10e14 GCs/kg by facial vein injection. All treated animals (n=6) died earlier than the untreated SMA mice. Yellowing skin and hepatocyte damage was found in vector treated animals at day 8 post injection (FIGS. 7A-7C). SMN1 overexpression was confirmed in the liver (FIG. 7D).

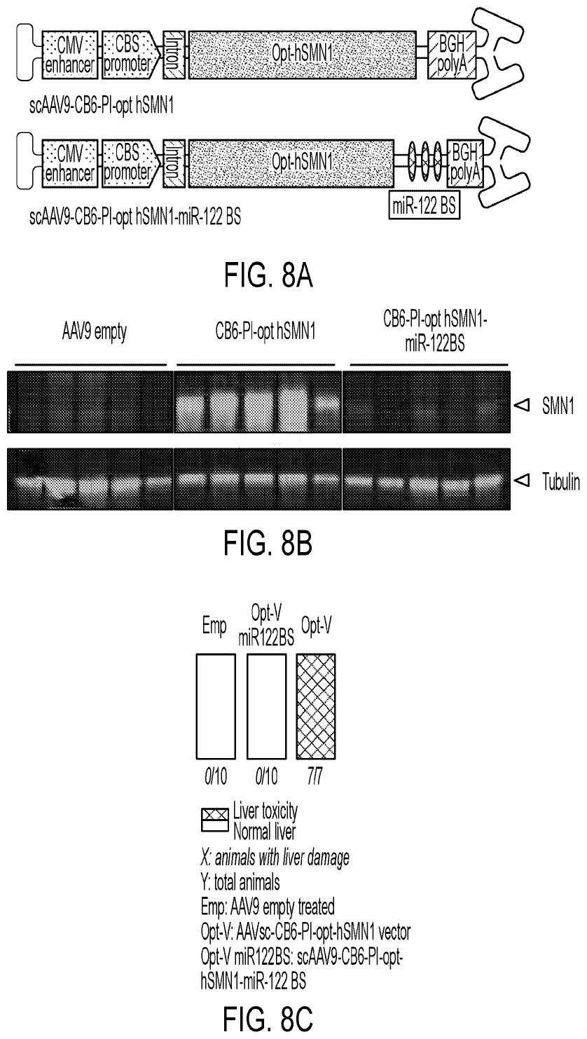

Further, whether abrogation of SMN expression in liver would alleviate liver damage was tested. In order to de-target the rAAV from the liver, miR-122 binding site was engineered into CMVen/CB-co-hSMN1 (CB6-PI-opt hSMN1-miR-122 BS; SEQ ID NO: 9) and packaged into AAV9 capsid (scAAV9-CB6-PI-opt hSMN1-miR-122 BS) (FIG. 8A). rAAV9sc-CMVen/CB-co-hSMN1 and scAAV9-CB6-PI-opt hSMN1-miR-122 BS were injected to postnatal day 1 SMNdelta7 mice, respectively. Liver expression of SMN1 and liver toxicity was evaluated. The results showed that, by using miR-122 binding site in the AAV construct, the expression of SMN was abrogated from the liver (FIG. 8B), and no liver toxicity was observed in mice treated with scAAV9-CB6-PI-opt hSMN1-miR-122 BS (FIG. 8C).

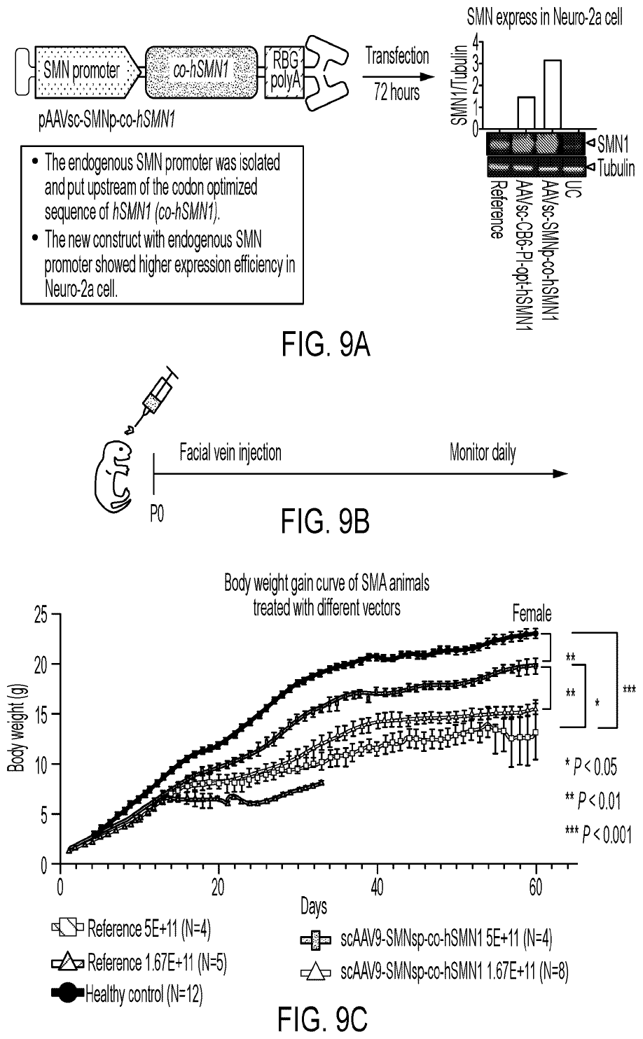

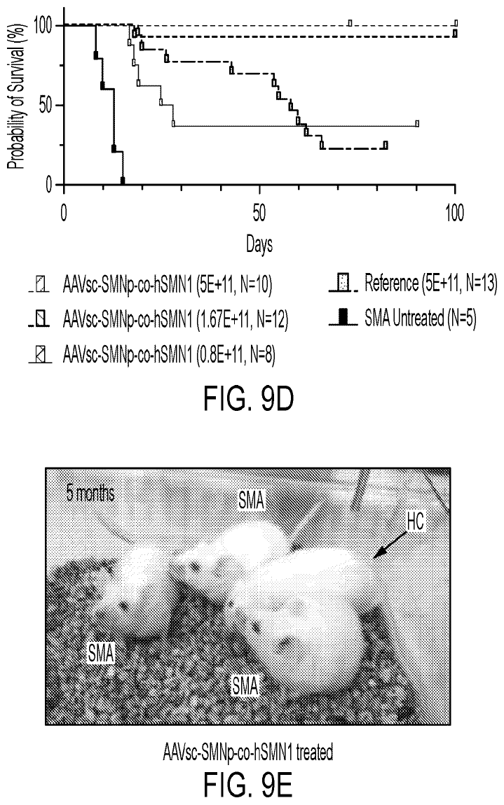

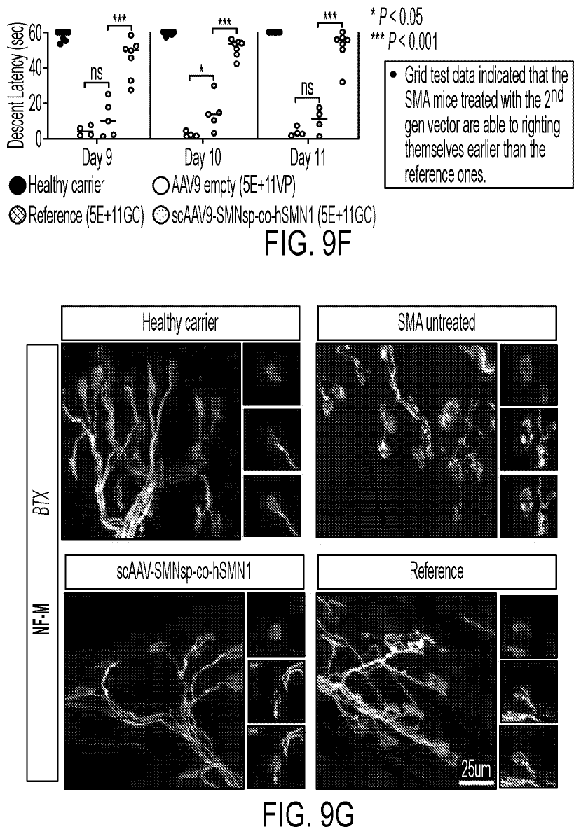

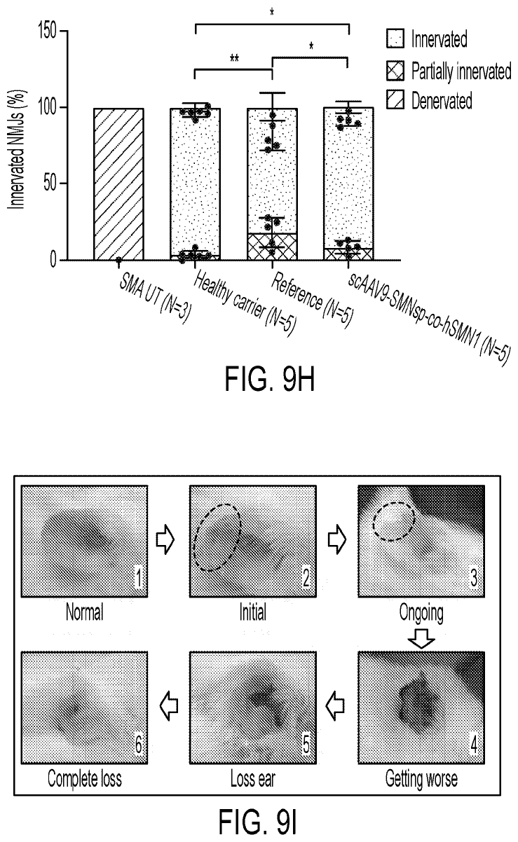

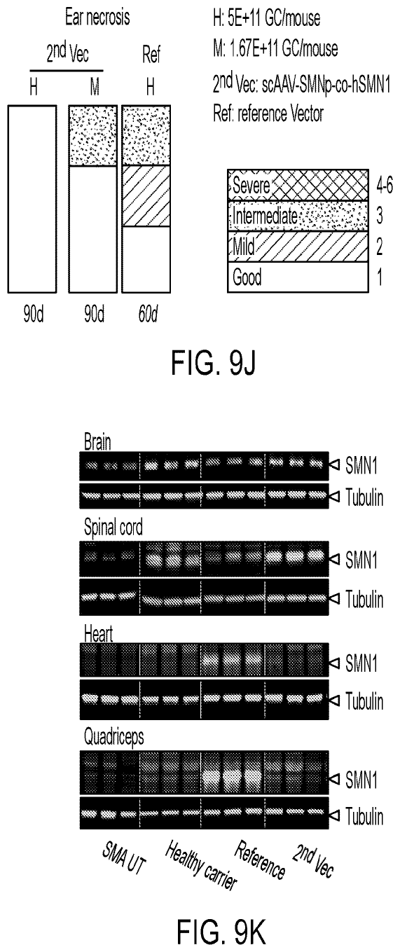

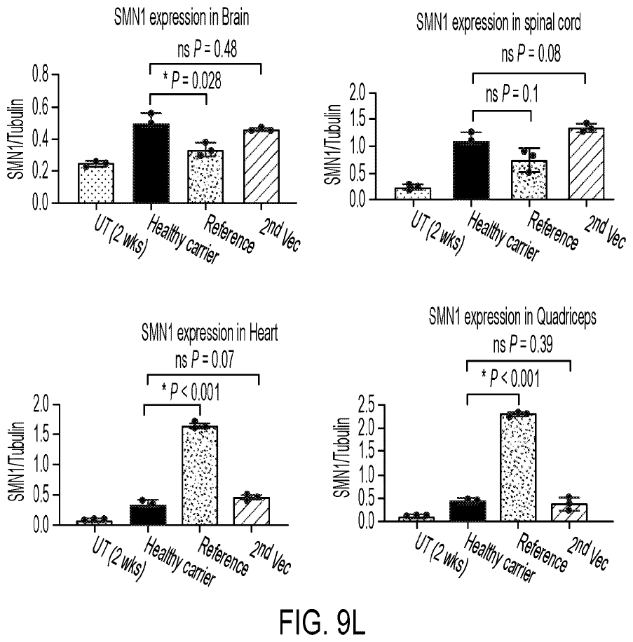

Further, it is possible that restoration of physiological levels of hSMN1 expression in the appropriate cell types with a low effective dose of rAAV can reduce liver toxicity. In order to achieve physiologically regulated hSMN1 expression, a second generation (2nd gen) scAAV9 expressing co-hSMN1 from an endogenous short hSMN1 promoter (SEQ ID NO: 5) was designed (SMN1sp-co-SMN1 (referred to as SMN1p-co-SMN1 in FIGS. 9D, 9E, and 9J); SEQ ID NO: 7). An scAAV9-CMVen/CB-opt-hSMN1 was used as a positive control. Neuro-2a cells were transfected with SMN1p-co-SMN1 or CMVen/CB-opt-hSMN1, and SMN1 expression was evaluated. The results show that the 2^{nd} generation construct with the endogenous SMN promoter showed higher expression efficiency in Neuro-2a cell (FIG. 9A).

Next, SMN1p-co-hSMN1 (SEQ ID NO: 7) was packaged into AAV9 capsid (scAAV9-SMNp-co-hSMN1) and injected neonatal SMA mice via facial vein injection (FIG. 9B). Another group of SMA mice with scAAV9-CMVen/CB-hSMN1 harboring the same expression cassette as used in Zolgensma® as the reference vector. The AAVsc-SMNsp-co-hSMN1 vector or the reference vector were injected into SMA mice via facial vein at P0 to set up a side-by-side animal experiment. Mice were monitored daily for body weight (FIG. 9C) and survival rate (FIG. 9D). The mice treated with AAVsc-SMNsp-co-hSMN1 gained more body weight than the reference vector group. The administration with AAVsc-SMNsp-co-hSMN1 vector significantly improved the life span of the SMA mice. The AAVsc-SMNsp-co-hSMN1 treated animal were healthy at 5 months old (FIG. 9E). Muscle function of the mice were also assessed. The treatment with AAVsc-SMNsp-co-hSMN1 achieved better restoration of the muscle functions than the reference group. Grid test data indicated that the SMA mice treated with AAVsc-SMNsp-co-hSMN1 were able to righting themselves earlier than the reference ones (FIG. 9F).

Effect of treatment at P0 on neuromuscular junction (NMJ) of young (P12) animals was also evaluated. Representative images from transverse abdominis (TVA) shows the treatment effect on the restoration of innervation in NMJs (FIG. 9G). The structures of neuromuscular junctions in the AAVsc-SMNsp-co-hSMN1-treated mice were restored close to that of wild-type mice, better than those of reference-treated SMA mice (FIG. 9H).

Moreover, the treatment with AAVsc-SMNsp-co-hSMN1 alleviated complication in SMA mice. Ear necrosis is one of complication usually found in this animal model. The severity of this complication was categorized into deferent disease levels (FIG. 9I). The AAVsc-SMNsp-co-hSMN1 treated animals showed less severity than the reference ones (FIG. 9J). The older of the animals the severer of the symptom. Data was collected from 90 days for the AAVsc-SMNp-co-hSMN1 treated animals, and 60 days for the reference treated animals.

The administration of AAVsc-SMNsp-co-hSMN1 in SMA mice achieved similar organ/tissue expression pattern with healthy carrier animals. Immunoblot assay indicated that the AAVsc-SMNp-co-hSMN1 preferentially express in central nervous system other than peripheral tissues. The expression pattern of this vector is close to the nature distribution of SMN as in healthy control animals. (FIGS. 9K-9L).

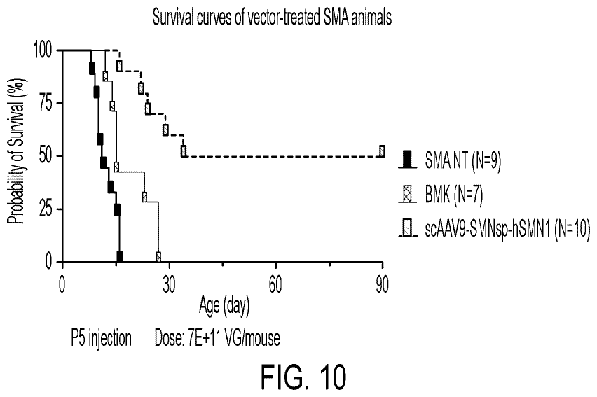

Further, SMA mice received AAVsc-SMNsp-co-hSMN1 or benchmark rAAV (BMK) at P5 were monitored for survival. The administration with scAAV9-SMNsp-hSMN1 (7E+11 VG/mouse) vector resulted in 50% survival of SMA mice up to 90 days post injection, whereas none of the SMA mice received the benchmark rAAV survived passed 30 days after injection (FIG. 10).

What is claimed is:

1. A recombinant adeno-associated virus (rAAV) vector comprising a transgene comprising an endogenous SMN1 promoter operably linked to a codon-optimized nucleic acid sequence encoding human SMN1, flanked by adeno-associated virus (AAV) inverted terminal repeats (ITRs).

2. The rAAV vector of claim 1, wherein the codon-optimized nucleic acid sequence comprises a nucleic acid sequence that is at least 70%, 80%, 90%, 95%, or 99% identical to the nucleic acid sequence set forth in SEQ ID NO: 1.

3. The rAAV vector of claim 1 or 2, wherein the codon-optimized nucleic acid sequence comprises or consists of the sequence set forth in SEQ ID NO: 1.

4. The rAAV vector of any one of claims 1 to 3, wherein the codon-optimized nucleic acid sequence does not comprise the nucleic acid sequence set forth in SEQ ID NO: 2.

5. The rAAV vector of any one of claims 1 to 3, wherein the human SMN1 comprises the amino acid sequence set forth in SEQ ID NO: 3.

6. The rAAV vector of any one of claims 1 to 5, wherein the endogenous SMN1 promoter is a human SMN1 promoter.

7. The rAAV vector of any one of claims 1 to 6, wherein the endogenous SMN1 promoter comprises a nucleic acid sequence that is at least 70%, 80%, 90%, 95%, or 99% identical to the nucleic acid sequence set forth in SEQ ID NO: 4 or 5.

8. The rAAV vector of any one of claims 1 to 7, wherein the endogenous SMN1 promoter comprises or consists of the nucleic acid sequence set forth in SEQ ID NO: 4.

9. The rAAV vector of any one of claims 1 to 7, wherein the endogenous SMN1 promoter comprises or consists of the nucleic acid sequence set forth in SEQ ID NO: 5.

10. The rAAV vector of any one of claims 1-9, further comprising one or more miR-122 binding sites.

11. The rAAV vector of claim 10, wherein the one or more miR-122 binding sites are placed between the codon-optimized nucleic acid sequence encoding human SMN1 and the 3′ ITR.

12. The rAAV vector of any one of claims 1 to 11, wherein at least one of the AAV ITRs is an AAV2 ITR.

13. The rAAV vector of any one of claims 1 to 12, wherein at least one of the AAV ITRs is a mutant ITR (mTR).

14. A vector comprising the rAAV vector of any one of claims 1 to 13.

15. The vector of claim 14, wherein the vector is a plasmid or a baculovirus vector.

16. A cell comprising the rAAV vector of any one of claims 1 to 13, or the vector of claim 14 or 15.

17. An isolated nucleic acid comprising the nucleic acid sequence set forth in any one of SEQ ID NO: 1, or SEQ ID NOs: 6-11.

18. The isolated nucleic acid of claim 17 further comprising an endogenous SMN1 promoter operably linked to the nucleic acid sequence.

19. The isolated nucleic acid of claim 18, wherein the endogenous SMN1 promoter is a human SMN1 promoter, optionally wherein the SMN1 promoter comprises or consists of the nucleic acid sequence set forth in SEQ ID NO: 4 or 5.

20. A recombinant adeno-associated virus (rAAV) comprising:
(i) a recombinant adeno-associated virus (rAAV) vector comprising a transgene comprising an endogenous SMN1 promoter operably linked to a codon-optimized nucleic acid sequence encoding human SMN1, flanked by adeno-associated virus (AAV) inverted terminal repeats (ITRs); and
(ii) at least one AAV capsid protein.

21. The rAAV of claim 20, wherein the codon-optimized nucleic acid sequence comprises a nucleic acid sequence that is at least 70%, 80%, 90%, 95%, or 99% identical to the nucleic acid sequence set forth in SEQ ID NO: 1.

22. The rAAV of claim 20 or 21, wherein the codon-optimized nucleic acid sequence comprises or consists of the sequence set forth in SEQ ID NO: 1.

23. The rAAV of any one of claims 20-22, wherein the codon-optimized nucleic acid sequence does not comprise the nucleic acid sequence set forth in SEQ ID NO: 2.

24. The rAAV of any one of claims 20-23, wherein the human SMN1 comprises the amino acid sequence set forth in SEQ ID NO: 3.

25. The rAAV of any one of claims 20-24, wherein the endogenous SMN1 promoter is a human SMN1 promoter.

26. The rAAV of any one of claims 20-25, wherein the endogenous SMN1 promoter comprises a nucleic acid sequence that is at least 70%, 80%, 90%, 95%, or 99% identical to the nucleic acid sequence set forth in SEQ ID NO: 4 or 5.

27. The rAAV of any one of claims 20-26, wherein the endogenous SMN1 promoter comprises or consists of the nucleic acid sequence set forth in SEQ ID NO: 4.

28. The rAAV of any one of claims 20-26, wherein the endogenous SMN1 promoter comprises or consists of the nucleic acid sequence set forth in SEQ ID NO: 5.

29. The rAAV of any one of claims 20-28, further comprising one or more miR-122 binding sites.

30. The rAAV of claim 29, wherein the one or more miR-122 binding sites are placed between the codon-optimized nucleic acid sequence encoding human SMN1 and the 3′ ITR.

31. The rAAV of any one of claims 20-30, wherein at least one of the AAV ITRs is an AAV2 ITR.

32. The rAAV of any one of claims 20-31, wherein at least one of the AAV ITRs is a mutant ITR (mTR).

33. The rAAV of any one of claims 20-32, wherein the rAAV is a self-complementary AAV (scAAV).

34. The rAAV of any one of claims 20-33, wherein the at least one AAV capsid protein is an AAV2, AAV3, AAV4, AAV5, AAV6, AAV7, AAV8, or AAV9 capsid protein, or a variant thereof.

35. The rAAV of any one of claims 20-34, wherein the at least one AAV capsid protein is an AAV9 capsid protein.

36. A pharmaceutical composition comprising the rAAV vector or rAAV of any one of the preceding claims, and a pharmaceutically acceptable excipient.

37. A method for delivering a transgene to a cell, the method comprising administering the rAAV vector of any one of claims 1 to 13, the rAAV of any one of claims 20-35, or the pharmaceutical composition of claim 36 to a cell.

38. The method of claim 37, wherein the cell is a mammalian cell.

39. The method of claim 37 or 38, wherein the cell is a human cell.

40. The method of any one of claims 37-39, wherein the cell is in a subject.

41. The method of claim 40, wherein the subject has or is suspected of having spinal muscular atrophy (SMA).

42. A method for preventing or treating spinal muscular atrophy (SMA) in a subject, the method comprising administering the rAAV vector of any one of claims 1 to 13, the rAAV of any one of claims 20-35, or the pharmaceutical composition of claim 36 to the subject.

43. The method of claim 42, wherein the subject is a mammal.

44. The method of claim 42 or 43, wherein the subject is a human.

45. The method of any one of claims 42-44, wherein the subject has one or more mutations in a SMN1 gene.

46. The method of any one of claims 42-45, wherein the administering comprises systemic injection or local injection.

47. The method of claim 46, wherein the systemic injection comprises intravenous injection.

48. The method of any one of claims 42-47, wherein the administering comprises injection to the central nervous system (CNS) of the subject.

49. The method of any one of claims 42-48, wherein the administration results in a reduced amount of dorsal root ganglion (DRG) toxicity in the subject relative to administration of an rAAV comprising a constative promoter operably linked to a wild-type SMN1-coding sequence to the subject.

50. The method of any one of claims 42-49, wherein the administration results in reduced liver toxicity in the subject relative to administration of an AAV vector comprising a constitutive promoter operably linked to a nucleic acid encoding human SMN1 to the subject.

51. The method of any one of claims 42-50, wherein the administration results in reduced complication associated with SMA.

52. The method of any one of claims 42-51, wherein the administration results in improved survival in the subject relative to administration of an AAV vector comprising a constitutive promoter operably linked to a nucleic acid encoding human SMN1 to the subject.

53. The method of claim 51, wherein the complication of SMA comprises pulmonary infections, spinal deformities (e.g., scoliosis, hip subluxations/dislocations), joint contractures, or respiratory failure.

54. A recombinant adeno-associated virus (rAAV) vector comprising a transgene comprising a promoter operably linked to a codon-optimized nucleic acid sequence encoding human SMN1, flanked by adeno-associated virus (AAV) inverted terminal repeats (ITRs).

55. The rAAV vector of claim 54, wherein the codon-optimized nucleic acid sequence comprises a nucleic acid sequence that is at least 70%, 80%, 90%, 95%, or 99% identical to the nucleic acid sequence set forth in SEQ ID NO: 1.

56. The rAAV vector of claim 54 or 55, wherein the codon-optimized nucleic acid sequence comprises or consists of the sequence set forth in SEQ ID NO: 1.

57. The rAAV vector of any one of claims 54-56, wherein the codon-optimized nucleic acid sequence does not comprise the nucleic acid sequence set forth in SEQ ID NO: 2.

58. The rAAV vector of any one of claims 54-57, wherein the human SMN1 comprises the amino acid sequence set forth in SEQ ID NO: 3.

59. The rAAV vector of any one of claims 54-58, wherein the promoter is a CB6 promoter.

60. The rAAV vector of any one of claim 54-59, wherein the rAAV vector further comprising a CMV enhancer.

61. The rAAV vector of any one of claims 54-60, further comprising one or more miR-122 binding sites.

62. The rAAV vector of claim 61, wherein the one or more miR-122 binding sites are placed between the codon-optimized nucleic acid sequence encoding human SMN1 and the 3′ ITR.

63. The rAAV vector of any one of claims 54-62, wherein at least one of the AAV ITRs is an AAV2 ITR.

64. The rAAV vector of any one of claims 54-63, wherein at least one of the AAV ITRs is a mutant ITR (mTR).

65. A vector comprising the rAAV vector of any one of claims 54-64.

66. The vector of claim 65, wherein the vector is a plasmid or a baculovirus vector.

67. A cell comprising the rAAV vector of any one of claims 54-64, or the vector of claim 65 or 66.

68. A recombinant adeno-associated virus (rAAV) comprising:
(a) a self-complementary rAAV genome comprising:
(i) a 5′ ITR
(ii) a human short SMN promoter comprising the nucleotide sequence of SEQ ID NO: 5;
(iii) a codon optimized nucleic acid sequence encoding SMN1 as set forth in SEQ ID NO: 1;
(iv) a poly A tail; and
(v) a 3′ ITR; and
(b) a AAV9 capsid protein.

69. The rAAV of claim 68, wherein the poly A tail is a rabbit globin poly A or a BGH poly A tail.

70. The rAAV of claim 68 or 69, further comprising one or more miR-122 binding sites.

71. A recombinant adeno-associated virus (rAAV) comprising:
(i) a recombinant adeno-associated virus (rAAV) vector of any one of claims 54 to 65, and
(ii) at least one AAV capsid protein.

72. The rAAV of claim 71, wherein the rAAV is a self-complementary AAV (scAAV).

73. The rAAV of claim 71 or 72, wherein the at least one AAV capsid protein is an AAV2, AAV3, AAV4, AAV5, AAV6, AAV7, AAV8, or AAV9 capsid protein, or a variant thereof.

74. The rAAV of any one of claims 71-73, wherein the at least one AAV capsid protein is an AAV9 capsid protein.

75. A method for preventing or treating spinal muscular atrophy (SMA) in a subject, the method comprising administering the rAAV vector of any one of claims 54-64, or the rAAV of any one of claims 71-74 to the subject.

76. The method of claim 75, wherein the subject is a mammal.

77. The method of claim 75 or 76, wherein the subject is a human.

78. The method of any one of claims 75-77, wherein the subject has one or more mutations in a SMN1 gene.

79. The method of any one of claims 75-78, wherein the administering comprises systemic injection or local injection.

80. The method of claim 79, wherein the systemic injection comprises intravenous injection.

81. The method of any one of claims 75-80, wherein the administering comprises injection to the central nervous system (CNS) of the subject.

82. The method of any one of claims 75-81, wherein the administration results in a reduced amount of dorsal root ganglion (DRG) toxicity in the subject relative to administration of an rAAV comprising a constative promoter operably linked to a wild-type SMN1-coding sequence to the subject.

83. The method of any one of claims 75-82, wherein the administration results in reduced liver toxicity in the subject relative to administration of an AAV vector comprising a constitutive promoter operably linked to a nucleic acid encoding human SMN1.

84. The method of any one of claims 75-83, wherein the administration results in reduced complication associated with SMA.

85. The method of any one of claims 74-83, wherein the administration results in improved survival in the subject relative to administration of an AAV vector comprising a constitutive promoter operably linked to a nucleic acid encoding human SMN1 to the subject.

86. The method of claim 84, wherein the complication of SMA comprises pulmonary infections, spinal deformities (e.g., scoliosis, hip subluxations/dislocations), joint contractures, or respiratory failure.
